(19) United States
(12) Patent Application Publication
(10) Pub. No.: US 2023/0008463 A1
(43) Pub. Date: Jan. 12, 2023

(54) INTELLIGENT SCREENING ALGORITHM AND AUTOMATIC UPGRADING SYSTEM FOR CONGENITAL HEART DISEASES OF NEWBORNS BASED ON BIG DATA

(71) Applicant: Children's Hospital of Fudan University, Shanghai (CN)

(72) Inventors: Guoying Huang, Shanghai (CN); Xiaojing Hu, Shanghai (CN); Chunyu Wang, Shanghai (CN); Chengqiang Zhang, Shanghai (CN)

(73) Assignee: Children's Hospital of Fudan University, Shanghai (CN)

(21) Appl. No.: 17/949,275

(22) Filed: Sep. 21, 2022

(30) Foreign Application Priority Data
Mar. 16, 2022 (CN) 202210260708.5

(51) Int. Cl. G16H 50/30; G16H 50/70

(52) U.S. Cl. CPC G16H 50/30; G16H 50/70

(57) ABSTRACT

The present disclosure discloses an intelligent screening algorithm and automatic upgrading system for congenital heart diseases of newborns based on big data. The key point of the technical solution is as follows: The intelligent screening algorithm and automatic upgrading system includes a heart sound data module, a heart sound data processing module, a blood oxygen data module, a blood oxygen data processing module, a network upgrading module, a database, an intelligent analysis module and a congenital heart disease evaluation module; the heart sound data module is configured to acquire various data of heart sounds of a newborn for centralized processing; and the heart sound data processing module is configured to process the data in the heart sound data module and extract heart sound feature parameters.

### TECHNICAL FIELD

The present disclosure relates to the technical field of screening of congenital heart diseases, in particular to an intelligent screening algorithm and automatic upgrading system for congenital heart diseases of newborns based on big data.

### BACKGROUND

Congenital heart disease is the most common type of congenital malformations, accounting for about 28% of all kinds of congenital malformations. Congenital heart disease refers to an abnormal anatomical structure caused by a forming dysfunction or abnormal development of the heart and great vessels during the period of embryonic development, or a failure of closure channels that should be automatically closed after birth (it is normal for infants). The incidence of congenital heart diseases should not be underestimated, accounting for 0.4% to 1% of living babies.

At present, congenital heart diseases of newborns have always been screened manually. With the development of an artificial intelligence technology, the digital upgrading of traditional medical devices has continuously promoted the development of clinical medicine. The study on an existing “two-indicator” intelligent screening apparatus prototype for congenital heart diseases of newborns preliminarily achieves synchronous and automatic collection and intelligent identification of blood oxygen and heart sounds of two parts. However, this prototype has many defects in efficiency and accuracy of intelligent identification of physiological signals. The algorithm needs to be improved on the basis of a lot of clinical screening data to make an intelligent screening apparatus more intelligent and accurate.

Heart sound is a random non-stationary signal that appears periodically with the contraction and relaxation of the heart, and is important physiological information that reflects the state of the cardiovascular system. Heart sound is a weak physiological signal. In an acquisition process, a heart sound will be affected by environmental noise, power frequency noise, frication sounds and breath sounds. A collected heart sound signal needs to be filtered and denoised before extraction of feature parameters. A digital filter, i.e. a low-pass filter, is used at the earliest, but this method will attenuate or lose high-frequency effective components, especially a noise component that indicates a cardiovascular disease, of a heart sound while filtering out high-frequency noise. Compared with traditional Fourier transform and Gabor transform, wavelet transform has the ability to refine local features in both time and frequency domains, and is particularly suitable for the analysis of non-stationary signals such as heart sounds. In general, wavelet threshold denoising is mainly divided into three steps: wavelet multi-scale decomposition, quantization threshold processing of wavelet coefficients at respective scales, and wavelet reconstruction. In order to achieve a satisfactory denoising effect, the determination and selection of all parameters in a wavelet algorithm, such as a wavelet basis function, the number of decomposition layers, and a threshold function, are very important. In terms of the wavelet basis function, dbN, symN, coifN, etc. are commonly used. In terms of the threshold function, a local maximum threshold method, a global threshold method, an adaptive threshold method, etc. can be selected; and the number of decomposition layers involves balancing the computing amount and the denoising effect. Four to seven layers are generally selected. At present, the wavelet denoising research on heart sound signals is carried out around the three aspects, but there is no general selection theory or principle, and each scholar selects parameters according to their own study purposes.

For cardiophony, products that have appeared on the market currently include the “StethCloud” system for detection and preliminary diagnosis of childhood pneumonia by the University of Melbourne, the digital stethoscope The One developed by Thinkslabs Medical in the United States, the Eko Core intelligent auscultation device developed by Eko Devices in California, the Radish smart stethoscope of Chengdu Radish Technology, the ChildCare cloud smart stethoscope of Shanghai Tuoxiao Intelligent Technology Co., Ltd., and cardiovascular real-time detection systems introduced by Xi'an Jiaotong University in China on the basis of a microfluidic technology and a mobile phone APP, which are all attempts and applications in this field. In addition, the Guo Xingming and Xiao Shouzhong team of Chongqing University and the Cheng Xiefeng team of Nanjing University of Posts and Telecommunications in China have carried out systematic research and development from single machines to systems and from algorithms to products. Chongqing University introduced an electronic stethoscope system, which collects heart sounds on the basis of a condenser microphone, achieves storage, transmission and playback of heart sound signals by combining Bluetooth, organic light-emitting diode (OLED), secure digital (SD) card, etc., and achieves intelligent analysis and database management of heart sounds based on a personal computer (PC). Nanjing University of Posts and Telecommunications designed a four-channel stethoscope for congenital heart disease screening. This four-channel stethoscope can monitor 4 auscultation areas at the same time, which improves the operation efficiency. Due to various reasons, these products have not been promoted on the market, and only stay at the level of reports and academic papers.

### SUMMARY

The present disclosure aims to provide an intelligent screening algorithm and automatic upgrading system for congenital heart diseases of newborns based on big data, so as to solve the problems mentioned in the above-mentioned background.

In order to achieve the above purposes, the present disclosure provides the following technical solution:

An intelligent screening algorithm and automatic upgrading system for congenital heart diseases of newborns based on big data includes a heart sound data module, a heart sound data processing module, a blood oxygen data module, a blood oxygen data processing module, a network upgrading module, a database, an intelligent analysis module and a congenital heart disease evaluation module;

wherein the heart sound data module is configured to acquire various data of heart sounds of a newborn for centralized processing;

the heart sound data processing module is configured to process the data in the heart sound data module and extract heat sound feature parameters;

the blood oxygen data module is configured to acquire various data of blood oxygen of the newborn for centralized processing;

the blood oxygen data processing module is configured to process the data in the blood oxygen data module and extract blood oxygen feature parameters;

the network upgrading module is configured to receive the heart sound feature parameters and the blood oxygen feature parameters and update in real time internal data of the database;

the database is in communication connection with a network and configured to update in real the feature parameters of the congenital heart disease ofthe newborn;

an artificial neural network algorithm, a support vector machine algorithm, the hidden Markov model (HMI) algorithm and the K-nearest neighbor algorithm are respectively built in the intelligent analysis module; the intelligent analysis module analyzes the heart sound feature parameters and the blood oxygen feature parameters on the basis of big data analysis for congenital heart disease screening, so as to distinguish signals from a healthy person and a patient;

the congenital heart disease evaluation module is configured to analyze and evaluate a congenital heart disease screening result of the intelligent analysis module.

Preferably, the heart sound data processing module includes a heart sound wavelet denoising unit, an envelope extraction unit and a segmentation unit; the heart sound wavelet denoising unit performs wavelet transform on a noisy heart sound signal, processes, in a certain way, a wavelet coefficient obtained by the transform to remove noise contained in the signal, and performs inverse wavelet transform on the processed wavelet coefficient to obtain a denoised signal; the envelope extraction unit is configured to process a heart sound signal to obtain an envelope data point of the heart sound signal, and perform, on the basis of a filter, smoothing on the envelope data point of the heart sound signal; and the segmentation unit is configured to segment the heart sound signal.

Preferably, a blood oxygen wavelet denoising unit is arranged in the blood oxygen data processing module; and the blood oxygen wavelet denoising unit is configured to denoise a noisy blood oxygen signal.

Preferably, the network upgrading module includes a network connection unit and an update detection unit; the network connection unit is configured to establish a communication connection between the database and Internet big data through a network; and the update detection unit is configured to detect in real time data parameters of the congenital heart diseases of the newborns in the Internet big data.

Preferably, a firewall unit is arranged in the database, and the firewall unit is configured to defense hacker attack.

Preferably, the HM3/I algorithm includes a direct computing method, a forward algorithm and a backward algorithm.

Preferably, the database includes a data classification unit and a data inquiry unit; the data classification unit is configured to classify data in terms of familiarity; and the data inquiry unit is configured to inquire data information by means of entering key words.

Preferably, the intelligent analysis module further includes a heart murmur grading unit and a blood oxygen value unit; the heart murmur grading unit is configured to classify heart murmur; and the blood oxygen value unit is configured to calculate a specific numerical value of blood oxygen.

Preferably, each of the heart sound data processing module and the blood oxygen data processing module is provided with a signal classification unit; and the signal classification unit is configured to identify murmur and hypoxemia and establish a relationship between a two-indicator result and a corresponding congenital heart disease.

Preferably, the intelligent analysis module classifies the heart murmur using the Lasso algorithm and calculates a classification result; the formula ofthe Lasso algorithm is as follows:

$$H ( X ) = {arg min}_{β ∈ {R}^{P}} {‖ y - X β ‖}^{2} + λ ‖ β ‖$$

where R is a set of all real numbers; RP represents a p-dimensional vector; each component

$${arg min}_{β ∈ {R}^{P}} {‖ y - X β ‖}^{2}$$

is a real number; β is a related coefficient; is the least squares term; X represents an input result of each classifier; y represents a desired result; and λ represents a coefficient of regularization.

Compared with the prior art, the present disclosure has the following beneficial effects.

In the intelligent screening algorithm and automatic upgrading system for congenital heart diseases of newborns based on big data, an intelligent computing network between blood oxygen and heart sound waveform feature parameters and a congenital heart disease screening result can be established using a deep neural network on the basis of big data analysis, thus fmally achieving intelligent screening of congenital heart diseases of newborns and continuously improving the accuracy of screening. An intelligent screening apparatus is more intelligent and accurate by means of building the artificial neural network algorithm, the support vector machine algorithm, the HMM algorithm and the K-nearest neighbor algorithm in the intelligent analysis module; and meanwhile, continuous analysis and continuous update iteration can be performed according to continuous accumulation data, which finally achieves integration, automation, intelligence and homogenization of “two-indicator” screening of congenital heart diseases of newborns at high accuracy.

The intelligent screening algorithm and automatic upgrading system is of great significance for timely diagnosis of congenital heart diseases of newborns and effective reduction of the infant mortality rate and the long-term social and medical burden related to diseases, can be applied to screening of countrywide congenital heart diseases of newborns, is of great significance for standardizing screening technologies, improving the screening accuracy, reducing the training cost and the like, and fmally achieves integration, automation, intelligence and homogenization of a “two-indicator” screening process of the congenital heart diseases of the newborns, promotes the popularization and application of the “two-indicator” method in China, particularly in countries and underdeveloped areas, and makes contributions to improving the population quality of the newborns in China and realizing the great vision of increasing the average life expectancy by one year old in the 14th Five-Year Plan of China.

### BRIEF DESCRIPTION OF THE DRAWINGS

FIG. 1 is a structural block diagram of the present disclosure.

### DETAILED DESCRIPTION OF THE EMBODIMENTS

The technical solutions in the embodiments of the present disclosure will be described clearly and completely below in combination with the accompanying drawings of the embodiments of the present disclosure. Apparently, the described embodiments are only part of the embodiments of the present disclosure, not all embodiments. All other embodiments obtained by those of ordinary skill in the art based on the embodiments in the present disclosure without creative work shall fall within the protection scope of the present disclosure.

### Embodiment 1

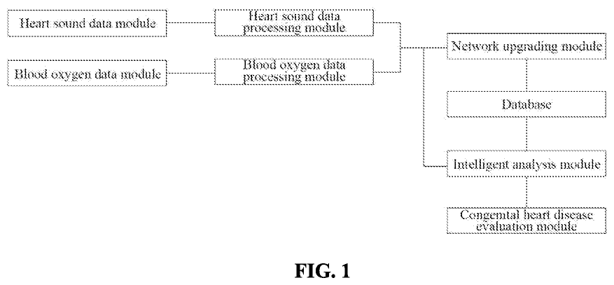

Referring to FIG. 1, the present disclosure provides an intelligent screening algorithm and automatic upgrading system for congenital heart diseases of newborns based on big data. The technical solution is as follows: the intelligent screening algorithm and automatic upgrading system includes a heart sound data module, a heart sound data processing module, a blood oxygen data module, a blood oxygen data processing module, a network upgrading module, a database, an intelligent analysis module and a congenital heart disease evaluation module.

The heart sound data module is configured to acquire various data of heart sounds of a newborn for centralized processing.

The heart sound data processing module is configured to process the data in the heart sound data module and extract heat sound feature parameters.

The blood oxygen data module is configured to acquire various data of blood oxygen of the newborn for centralized processing.

The blood oxygen data processing module is configured to process the data in the blood oxygen data module and extract blood oxygen feature parameters.

The network upgrading module is configured to receive the heart sound feature parameters and the blood oxygen feature parameters and update in real time internal data of the database.

The database is in communication connection with a network and configured to update in real the feature parameters of the congenital heart disease of the newborn.

An artificial neural network algorithm, a support vector machine algorithm, the HMM algorithm and the K-nearest neighbor algorithm are respectively built in the intelligent analysis module; and the intelligent analysis module analyzes the heart sound feature parameters and the blood oxygen feature parameters on the basis of big data analysis for congenital heart disease screening, so as to distinguish signals from a healthy person and a patient.

The congenital heart disease evaluation module is configured to analyze and evaluate a congenital heart disease screening result of the intelligent analysis module.

In this embodiment, preferably, the heart sound data processing module includes a heart sound wavelet denoising unit, an envelope extraction unit and a segmentation unit; the heart sound wavelet denoising unit performs wavelet transform on a noisy heart sound signal, processes, in a certain way, a wavelet coefficient obtained by the transform to remove noise contained in the signal, and performs inverse wavelet transform on the processed wavelet coefficient to obtain a denoised signal; the envelope extraction unit is configured to process a heart sound signal to obtain an envelope data point of the heart sound signal, and perform, on the basis of a filter, smoothing on the envelope data point of the heart sound signal; and the segmentation unit is configured to segment the heart sound signal.

In this embodiment, preferably, a blood oxygen wavelet denoising unit is arranged in the blood oxygen data processing module; and the blood oxygen wavelet denoising unit is configured to denoise a noisy blood oxygen signal.

In this embodiment, preferably, the network upgrading module includes a network connection unit and an update detection unit; the network connection unit is configured to establish a communication connection between the database and Internet big data through a network; and the update detection unit is configured to detect in real time data parameters of the congenital heart diseases of the newborns in the Internet big data.

In this embodiment, preferably, a firewall unit is arranged in the database, and the firewall unit is configured to defense hacker attack.

In this embodiment, preferably, the HMM algorithm includes a direct computing method, a forward algorithm and a backward algorithm.

In this embodiment, preferably, the database includes a data classification unit and a data inquiry unit; the data classification unit is configured to classify data in terms of familiarity; and the data inquiry unit is configured to inquire data information by means of entering key words.

In this embodiment, preferably, the intelligent analysis module further includes a heart murmur grading unit and a blood oxygen value unit; the heart murmur grading unit is configured to classify heart murmur; and the blood oxygen value unit is configured to calculate a specific numerical value of blood oxygen.

In this embodiment, preferably, each of the heart sound data processing module and the blood oxygen data processing module is provided with a signal classification unit; and the signal classification unit is configured to identify murmur and hypoxemia and establish a relationship between a two-indicator result and a corresponding congenital heart disease.

### Embodiment 2

Referring to FIG. 1, the present disclosure provides an intelligent screening algorithm and automatic upgrading system for congenital heart diseases of newborns based on big data. The technical solution is as follows: the intelligent screening algorithm and automatic upgrading system includes a heart sound data module, a heart sound data processing module, a blood oxygen data module, a blood oxygen data processing module, a network upgrading module, a database, an intelligent analysis module and a congenital heart disease evaluation module.

The heart sound data module is configured to acquire various data of heart sounds of a newborn for centralized processing.

The heart sound data processing module is configured to process the data in the heart sound data module and extract heat sound feature parameters.

The blood oxygen data module is configured to acquire various data of blood oxygen of the newborn for centralized processing.

The blood oxygen data processing module is configured to process the data in the blood oxygen data module and extract blood oxygen feature parameters.

The network upgrading module is configured to receive the heart sound feature parameters and the blood oxygen feature parameters and update in real time internal data of the database.

The database is in communication connection with a network and configured to update in real the feature parameters of the congenital heart disease of the newborn.

An artificial neural network algorithm, a support vector machine algorithm, the HMM algorithm and the K-nearest neighbor algorithm are respectively built in the intelligent analysis module; and the intelligent analysis module analyzes the heart sound feature parameters and the blood oxygen feature parameters on the basis of big data analysis for congenital heart disease screening, so as to distinguish signals from a healthy person and a patient.

The congenital heart disease evaluation module is configured to analyze and evaluate a congenital heart disease screening result of the intelligent analysis module.

In this embodiment, preferably, the heart sound data processing module includes a heart sound wavelet denoising unit, an envelope extraction unit and a segmentation unit; the heart sound wavelet denoising unit performs wavelet transform on a noisy heart sound signal, processes, in a certain way, a wavelet coefficient obtained by the transform to remove noise contained in the signal, and performs inverse wavelet transform on the processed wavelet coefficient to obtain a denoised signal; the envelope extraction unit is configured to process a heart sound signal to obtain an envelope data point of the heart sound signal, and perform, on the basis of a filter, smoothing on the envelope data point of the heart sound signal; and the segmentation unit is configured to segment the heart sound signal.

In this embodiment, preferably, a blood oxygen wavelet denoising unit is arranged in the blood oxygen data processing module; and the blood oxygen wavelet denoising unit is configured to denoise a noisy blood oxygen signal.

In this embodiment, preferably, the network upgrading module includes a network connection unit and an update detection unit; the network connection unit is configured to establish a communication connection between the database and Internet big data through a network; and the update detection unit is configured to detect in real time data parameters of the congenital heart diseases of the newborns in the Internet big data.

In this embodiment, preferably, a firewall unit is arranged in the database, and the firewall unit is configured to defense hacker attack.

In this embodiment, preferably, the HMM algorithm includes a direct computing method, a forward algorithm and a backward algorithm.

In this embodiment, preferably, the database includes a data classification unit and a data inquiry unit; the data classification unit is configured to classify data in terms of familiarity; and the data inquiry unit is configured to inquire data information by means of entering key words.

In this embodiment, preferably, the intelligent analysis module further includes a heart murmur grading unit and a blood oxygen value unit; the heart murmur grading unit is configured to classify heart murmur; and the blood oxygen value unit is configured to calculate a specific numerical value of blood oxygen.

In this embodiment, preferably, each of the heart sound data processing module and the blood oxygen data processing module is provided with a signal classification unit; and the signal classification unit is configured to identify murmur and hypoxemia and establish a relationship between a two-indicator result and a corresponding congenital heart disease.

In this embodiment, preferably, the intelligent analysis module classifies the heart murmur using the Lasso algorithm and calculates a classification result; the formula of the Lasso algorithm is as follows:

$$H ( X ) = {arg min}_{β ∈ {R}^{P}} {‖ y - X β ‖}^{2} + λ ‖ β ‖$$

where R is a set of all real numbers; RP represents a p-dimensional vector; each component is a real number; β is a related coefficient;

$${arg min}_{β ∈ {R}^{P}} {‖ y - X β ‖}^{2}$$

is the least squares term; X represents an input result of each classifier; y represents a desired result; and λ represents a coefficient of regularization.

The working principle and use flow of the present disclosure are as follows:

During use of the intelligent screening algorithm and automatic upgrading system for the congenital heart diseases of newborns based on the big data, the blood oxygen and heart sound data of a newborn are collected through the heart sound data module and the blood oxygen data module; the heart sound feature parameters and the blood oxygen feature parameters are extracted respectively through the heart sound data processing module and the blood oxygen data processing module; the network upgrading module is configured to receive the heart sound feature parameters and the blood oxygen feature parameters to update in real time the internal data of the database; the heart sound feature parameters and the blood oxygen feature parameters are analyzed on the basis of big data analysis for congenital heart disease screening, so as to distinguish signals from a healthy person and a patient; and the congenital heart disease screening result is obtained by analysis by the congenital heart disease evaluation module.

Although the embodiments of the present disclosure have been shown and described, it will be understood by those of ordinary skill in the art that various changes, modifications, substitutions, and variations can be made to these embodiments without departing from the principle and spirit of the present disclosure. The scope of the present disclosure is defined by the attached claims and their equivalents.

What is claimed is:

1. An intelligent screening algorithm and automatic upgrading system for congenital heart diseases of newborns based on big data, comprising a heart sound data module, a heart sound data processing module, a blood oxygen data module, a blood oxygen data processing module, a network upgrading module, a database, an intelligent analysis module and a congenital heart disease evaluation module;
wherein the heart sound data module is configured to acquire various data of heart sounds of a newborn for centralized processing;
the heart sound data processing module is configured to process the data in the heart sound data module and extract heat sound feature parameters;
the blood oxygen data module is configured to acquire various data of blood oxygen of the newborn for centralized processing;
the blood oxygen data processing module is configured to process the data in the blood oxygen data module and extract blood oxygen feature parameters;
the network upgrading module is configured to receive the heart sound feature parameters and the blood oxygen feature parameters and update in real time internal data of the database;
the database is in communication connection with a network and configured to update in real the feature parameters of the congenital heart disease of the newborn;
an artificial neural network algorithm, a support vector machine algorithm, the hidden Markov model (HMM) algorithm and the K-nearest neighbor algorithm are respectively built in the intelligent analysis module; the intelligent analysis module analyzes the heart sound feature parameters and the blood oxygen feature parameters on the basis of big data analysis for congenital heart disease screening, so as to distinguish signals from a healthy person and a patient;
the congenital heart disease evaluation module is configured to analyze and evaluate a congenital heart disease screening result of the intelligent analysis module.

2. The intelligent screening algorithm and automatic upgrading system for the congenital heart diseases of the newborns based on the big data according to claim 1, wherein the heart sound data processing module comprises a heart sound wavelet denoising unit, an envelope extraction unit and a segmentation unit; the heart sound wavelet denoising unit performs wavelet transform on a noisy heart sound signal, processes, in a certain way, a wavelet coefficient obtained by the transform to remove noise contained in the signal, and performs inverse wavelet transform on the processed wavelet coefficient to obtain a denoised signal; the envelope extraction unit is configured to process a heart sound signal to obtain an envelope data point of the heart sound signal, and perform, on the basis of a filter, smoothing on the envelope data point of the heart sound signal; and the segmentation unit is configured to segment the heart sound signal.

3. The intelligent screening algorithm and automatic upgrading system for the congenital heart diseases of the newborns based on the big data according to claim 1, wherein a blood oxygen wavelet denoising unit is arranged in the blood oxygen data processing module; and the blood oxygen wavelet denoising unit is configured to denoise a noisy blood oxygen signal.

4. The intelligent screening algorithm and automatic upgrading system for the congenital heart diseases of the newborns based on the big data according to claim 1, wherein the network upgrading module comprises a network connection unit and an update detection unit; the network connection unit is configured to establish a communication connection between the database and Internet big data through a network; and the update detection unit is configured to detect in real time data parameters of the congenital heart diseases of the newborns in the Internet big data.

5. The intelligent screening algorithm and automatic upgrading system for the congenital heart diseases of the newborns based on the big data according to claim 1, wherein a firewall unit is arranged in the database, and the firewall unit is configured to defense hacker attack.

6. The intelligent screening algorithm and automatic upgrading system for the congenital heart diseases of the newborns based on the big data according to claim 1, wherein the HMM algorithm comprises a direct computing method, a forward algorithm and a backward algorithm.

7. The intelligent screening algorithm and automatic upgrading system for the congenital heart diseases of the newborns based on the big data according to claim 1, wherein the database comprises a data classification unit and a data inquiry unit; the data classification unit is configured to classify data in terms of familiarity; and the data inquiry unit is configured to inquire data information by means of entering key words.

8. The intelligent screening algorithm and automatic upgrading system for the congenital heart diseases of the newborns based on the big data according to claim 1, wherein the intelligent analysis module further comprises a heart murmur grading unit and a blood oxygen value unit; the heart murmur grading unit is configured to classify heart murmur; and the blood oxygen value unit is configured to calculate a specific numerical value of blood oxygen.

9. The intelligent screening algorithm and automatic upgrading system for the congenital heart diseases of the newborns based on the big data according to claim 1, wherein each of the heart sound data processing module and the blood oxygen data processing module is provided with a signal classification unit; and the signal classification unit is configured to identify murmur and hypoxemia and establish a relationship between a two-indicator result and a corresponding congenital heart disease.

10. The intelligent screening algorithm and automatic upgrading system for the congenital heart diseases of the newborns based on the big data according to claim 8, wherein the intelligent analysis module classifies the heart murmur using the Lasso algorithm and calculates a classification result;
the formula of the Lasso algorithm is as follows:
$$H ( X ) = {arg min}_{β ∈ {R}^{P}} {‖ y - X β ‖}^{2} + λ ‖ β ‖$$
where R is a set of all real numbers; RP represents a p-dimensional vector; each component is a real number; β is a related coefficient;
$${arg min}_{β ∈ {R}^{P}} {‖ y - X β ‖}^{2}$$
is the least squares term; X represents an input result of each classifier; y represents a desired result; and λ represents a coefficient of regularization.
